(19) United States
(12) Patent Application Publication
(10) Pub. No.: US 2018/0148742 A1
(43) Pub. Date: May 31, 2018

(54) METHODS AND COMPOSITIONS FOR ETHANOL PRODUCTION

(71) Applicant: Syngenta Participations AG, Basel (CH)

(72) Inventors: Vijay Singh, Urbana, IL (US); Deepak Kumar, Urbana, IL (US)

(73) Assignee: Syngenta Participations AG, Basel (CH)

(21) Appl. No.: 15/804,404

(22) Filed: Nov. 6, 2017

Related U.S. Application Data
(60) Provisional application No. 62/427,224, filed on Nov. 29, 2016.

(51) Int. Cl. C12P 7/10; C07C 31/08; C07C 29/14; C12N 9/26; C12N 9/44; C12N 9/92; C12F 3/00; C12C 11/00; C12C 7/04

(52) U.S. Cl. CPC C12P 7/10; C07C 31/08; C07C 29/14; C12N 9/2411; C12C 7/04; C12N 9/92; C12F 3/00; C12C 11/003; C12N 9/2457

(57) ABSTRACT

The conventional corn dry grind ethanol production process requires the addition of exogenous alpha and glucoamylase enzymes to break down starch into glucose, which is fermented to ethanol by yeast. The present invention describes use of new genetically engineered corn and yeast, which can eliminate or minimize the use of these external enzymes, improve the economics and process efficiencies, and simplify the process.

### RELATED APPLICATION INFORMATION

This application claims the benefit of U.S. Provisional Application No. 62/427,224 filed 29 Nov. 2016, the content of which is incorporated by reference herein in its entirety.

### STATEMENT REGARDING ELECTRONIC SUBMISSION OF A SEQUENCE LISTING

A Sequence Listing in the ASCII text format, submitted under 37 CFR § 1.821, entitled “81160 Sequence Listing_ST25.txt”, 729 bytes in size, generated on Nov. 3, 2017 and filed via EFS-WEB is provided in lieu of a paper copy. This Sequence Listing is hereby incorporated by reference into the specification for its disclosures.

### FIELD OF THE INVENTION

The invention relates to methods for the production of ethanol from plant biomass.

### BACKGROUND OF THE INVENTION

Due to increasing population and industrialization, global energy demand has increased steadily over the last few decades, and currently about 80% of this energy is derived from non-renewable fossil fuel supplies [1]. The transportation sector is one of the major consumers of fossil fuels in the United States [2]. The concerns about depleting fossil fuel resources and the negative environmental impacts from their suggests the need to identify and develop renewable and sustainable energy sources. Bioethanol is considered the most promising renewable transportation fuel, which can be produced in significant quantities from the fermentation of sugars obtained from starch, sugary or cellulosic materials. The United States is the biggest bioethanol producer in world with about a 14.3 billion gallon (54.1 billion liters; 58% of world production) production in year 2014 [3]. Most of the ethanol in the United States is produced from corn using a dry grind or wet milling process. Dry grind is the most commonly used method for corn ethanol production [4]. In year 2014, about 5.4 billion bushels (25.4 kg in one bushel) of corn (37.8% of total production) was processed in the dry-grind industry [5].

FIG. 1 illustrates the major steps used during a conventional laboratory scale dry-grind process. The ground corn and water slurry is liquefied using alpha-amylase enzymes at high temperature to convert starch into dextrins. The dextrins are further converted to glucose using glucoamylase (GA) enzymes during a saccharification process, which glucose is fermented to ethanol by yeast. Currently these alpha and glucoamylase enzymes are added externally in liquid form during the liquefaction and saccharification process, respectively. Saccharification and fermentation are performed in a single step in the same reactor by a process known as simultaneous saccharification and fermentation (SSF). Ethanol is recovered from the fermentation broth using a distillation process. The remaining non-carbohydrate fractions from corn (germ, fiber, and protein) are recovered as a coproduct called distillers dried grains with solubles (DDGS) at the end of the process.

Over the last few decades, several advances have been made to improve the ethanol yields and profitability of the dry grind process, including modifications in the production process [6], recovery of high-value co-products [5,7], use of advanced enzymes [8-9], and use of high-yield corn varieties [10].

The present invention provides new methods and compositions for the production of ethanol from plant biomass.

### SUMMARY OF THE INVENTION

An aspect of the invention provides a method of making ethanol comprising the steps of: a) combining milled plant material comprising at least about 0.1% of plant material from a transgenic plant expressing alpha-amylase with a liquid to form a mash; b) hydrolyzing starch in the mash to form a liquefact; c) fermenting the liquefact in the presence of a yeast expressing glucoamylase to form a beer; and d) distilling the beer to produce ethanol.

In embodiments, the transgenic plant expressing alpha-amylase is a transgenic corn plant comprising the genotype of corn event 3272.

In representative embodiments, the plant material is corn grain.

According to any of the preceding embodiments, the milled plant material can comprise an admix of about 0.1% to about 100% of the transgenic corn plant comprising the genotype of corn event 3272 and about 99.1% to 0% of plant material not comprising the genotype of corn event 3272. In embodiments, the milled plant material comprises at least about 5% to about 50%, optionally about 5% to about 25%, of the transgenic corn plant comprising the genotype of corn event 3272.

In embodiments, no exogenous alpha-amylase is added in addition to that present in the plant material from the transgenic corn plant comprising the genotype of corn event 3272.

In embodiments, the genotype of corn event 3272 comprises the nucleotide sequence set forth in SEQ ID NO: 1 (ctgacgcggc caaacactga) and/or the nucleotide sequence set forth in SEQ ID NO: 2 (cacaatatat tcaagtcatc).

According to any of the preceding embodiments, the method can further comprise adding one or more starch-degrading enzymes selected from the group of alpha-amylase, pullulanase, alpha-glucosidase, glucoamylase, amylopullulanase, glucose isomerase, and combinations thereof.

Optionally, according to the methods of the invention, the amount of exogenous glucoamylase added is about 10% to about 60% of that added in a control method of making ethanol that does not comprise yeast expressing glucoamylase as in step (c).

In embodiments, no exogenous glucoamylase is added in addition to that from the yeast expressing glucoamylase.

According to any of the preceding embodiments, the yeast expressing glucoamylase can be a Saccharomyces spp, Candida spp., Schizosaccharomyces spp., Endomycopsis spp., Kluyveromyces spp., Pichia spp., Hanseniaspora spp., Trichoderma spp., Thermomyces spp., or any combination thereof. In embodiments, the yeast expressing glucoamylase can be Saccharomyces cerevisiae, Candida arabinofermentans, Candida shehatae, Candida utilis, Candida valida, Candida lyxosophila, Candida mogii, Candida santjackobensis, Candida succiphila, Candida sorboxylosa, Candida stellate, Candida tenuis, Candida veronae, Schizosaccharomyces pombe, Endomycopsis fibuligera, Kluyveromyces marxianu, Pichia mexicana, Pichia anomala, Pichia guilliermondii, Pichia stipitis, Hanseniaspora valbyensis Trichoderma reesei., Thermomyces lanuginosus, or any combination thereof.

According to any of the preceding embodiments, the liquid can comprise water.

According to any of the preceding embodiments, hydrolyzing starch in the mash can be performed at a pH of about pH 3.8 to about pH 5.0.

According to any of the preceding embodiments, the mash can have a percentage of solids of about 35% to about 60%, optionally at least about 37%.

According to any of the preceding embodiments, the mash can have a temperature that does not exceed about 90° C.

According to any of the preceding embodiments, after step (a) and prior to hydrolyzing the starch in the mash to form a liquefact (step (b)), the method can comprise allowing the mash to flow from initial combining to a heat exchanger at a rate which is at least 5% to at least 70%, optionally at least 20% to at least 55%, less than the rate of flow of beer from a beer well to a distiller.

According to any of the preceding embodiments, after step (a) and prior to hydrolyzing the starch in the mash to form a liquefact (step (b)), the method can comprise allowing the mash to flow from initial combining to a fermenter at a rate which is at least 5% to at least 70%, optionally at least 20% to at least 55%, less than the rate of flow of beer from a beer well to a distiller. Optionally, the method can further comprise the step of allowing the mash to flow out of a fermenter at a rate that is approximately the nameplate flow rate. In embodiments, the method comprises allowing the mash to flow from initial combining to a fermenter at a rate which is at least 20% to at least 55% less than the nameplate rate of flow.

This and other aspects of the invention are set forth in more detail in the description of the invention below.

### BRIEF DESCRIPTION OF THE DRAWINGS

FIG. 1 presents a schematic of a laboratory scale dry-grind corn process for ethanol production.

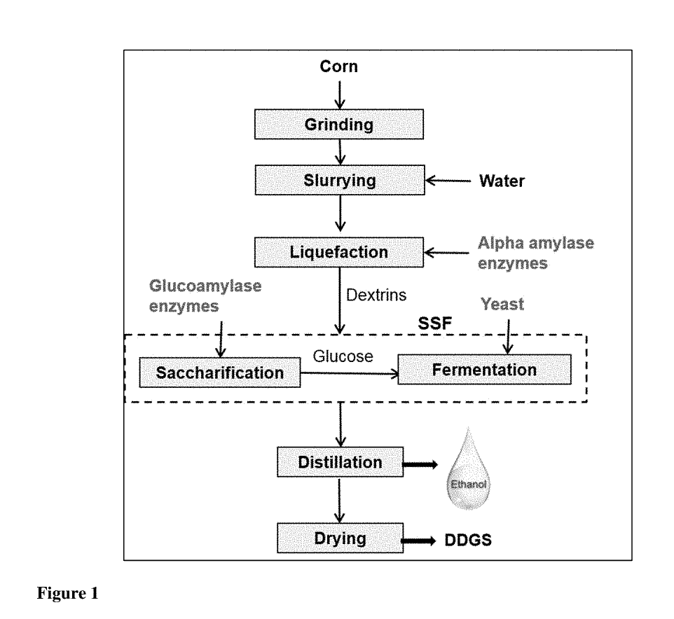

FIG. 2 illustrates the details of the vacuum-assisted fermentation system used in these studies.

FIG. 3 provides the comparison of ethanol concentrations (% v/v) and glucose concentrations (% w/v) during SSF of 100% dent and 15% amylase corn mix. The data points in the figure are means of triplicate runs and error bars represent standard deviations.

FIG. 4 illustrates the fermentation profile of dent corn mix during SSF by superior yeast at various GA loadings and conventional yeast. Solid lines refer to ethanol concentrations (% v/v) and dotted lines refer to glucose concentrations (% w/v). The data points in the figure are means of triplicate runs and error bars represent standard deviations.

FIG. 5 provides a comparison of glycerol concentration (% w/v) during SSF of dent corn between conventional yeast (control) and superior yeast at various GA loadings. The bars in the figure are means of triplicate runs and error bars represent standard deviations.

FIG. 6 illustrates the fermentation profile of amylase corn mix during SSF by superior yeast at various GA loadings and conventional yeast. The data points in the figure are means of triplicate runs and error bars represent standard deviations. Solid lines refer to ethanol concentrations (% v/v) and dotted lines refer to glucose concentrations (% w/v).

FIG. 7 provides a comparison of glycerol concentration (% w/v) during SSF of amylase corn mix among conventional yeast (control) and superior yeast at various GA loading. The bars in the figure are means of triplicate runs and error bars represent standard deviations.

FIG. 8 illustrates the effect of solid loadings on the ethanol concentration (% v/v) and glucose concentrations (% w/v) of amylase corn mix during SSF using superior yeast and 50% GA. The data points in the figure are means of triplicate runs and error bars represent standard deviations.

FIG. 9 illustrates the comparison of glucose concentrations (% w/v) and ethanol concentrations (% v/v) during fermentation of amylase corn mix using superior yeast and 50% GA between conventional and vacuum-assisted fermentation.

FIG. 10 illustrates the comparison of of glucose concentrations (% w/v) and ethanol concentrations (% v/v) during fermentation of amylase corn mix using superior yeast and 50% GA between conventional and vacuum-assisted fermentation.

### DETAILED DESCRIPTION OF THE INVENTION

The present invention will now be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like refers to variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

“Alpha-amylase” refers to an enzyme which cleaves or hydrolyzes internal alpha (1-4) glycosidic bonds in starch to produce smaller molecular weight maltodextrins. These smaller molecular weight maltodextrins include, but are not limited to, maltose, which is a disaccharide (i.e., a dextrin with a degree of polymerization of 2 or a DP2), maltotriose (a DP3), maltotetrose (a DP4), and other oligosaccharides. The enzyme alpha-amylase (EC 3.2.1.1) can also be referred to as 1,4-alpha-D-glucan glucanohydrolase or glycogenase. A variety of alpha-amylases are known in the art and are commercially available. An alpha-amylase can be from a fungal or bacterial origin and can be expressed in transgenic plants. The alpha-amylase can be thermostable.

“Glucoamylase” (also known as amyloglucosidase) refers to the enzyme that has the systematic name 1,4-alpha-D-glucan glucohydrolase (E.C. 3.2.1.3). Glucoamylase removes successive glucose units from the non-reducing ends of starch. A variety of glucoamylases are known in the art and are commercially available. For example, certain glucoamylases can hydrolyze both the linear and branched glucosidic linkages of starch, amylose, and amylopectin. Glucoamylase can be from a fungal origin and can be expressed in transgenic plants. The glucoamylase can be thermostable

As used herein the term “dextrose equivalents” or “dextrose equivalent” refers to the industry standard for measuring the concentration of total reducing sugars, calculated as glucose on a dry weight basis. Unhydrolyzed granular starch has a dextrose equivalent of virtually zero, whereas the dextrose equivalent of glucose is defined as 100.

The term “slurry” refers to a mixture of starch or a starch-containing material (e.g., milled corn) and an aqueous component, which can include, for example, water, de-ionized water, or a process water (i.e., backset, steam, condensate), or any combination thereof. The terms “slurry” and “mash” can be used interchangeably.

The term “admix” or “admixture” refers to a combination of elements. For example, an admix of plant material can refer to mixing of two or more plant materials together to form a mixture. It is possible to further define the admixture by indicating the percentage of one or more of the elements. The plant material can be comprised of both transgenic and non-transgenic plant material. Transgenic plant material can contain a heterologous transgene encoding an enzyme, an insect control gene, an herbicide tolerance gene, a phytase, a nematode control gene or any other transgenic gene. The transgenic material may be expressing more than one transgene.

As used herein the terms “liquefaction,” “liquefy,” “liquefact,” and variations thereof refer to the process or product of converting starch to soluble dextrinized substrates (e.g., smaller polysaccharides). “Liquefact” can also be referred to as “mash.”

The term “secondary liquefaction” refers to a liquefaction process that takes place after an initial period of liquefaction or after a jet cooking step of a multi-stage liquefaction process. The secondary liquefaction can involve a different temperature than a previous liquefaction step or can involve the addition of additional starch-digesting enzymes (e.g., alpha-amylase).

As used herein, the terms “saccharification” and “saccharifying” refer to the process of converting polysaccharides to dextrose monomers using enzymes. Saccharification can specifically refer to the conversion of polysaccharides in a liquefact. Saccharification products are, for example, glucose and other small (low molecular weight) oligosaccharides such as disaccharides (a DP2) and trisaccharides (a DP3).

“Fermentation” or “fermenting” refer to the process of transforming sugars from reduced plant material to produce alcohols (e.g., ethanol, methanol, butanol, propanol); organic acids (e.g., citric acid, acetic acid, itaconic acid, lactic acid, gluconic acid, propionate); ketones (e.g., acetone), amino acids (e.g., glutamic acid); gases (e.g., H₂ and CO₂), antibiotics (e.g., penicillin and tetracycline); enzymes; vitamins (e.g., riboflavin, B₁₂, beta-carotene); and/or hormones. Fermentation can include fermentations used in the consumable alcohol industry (e.g., beer and wine), dairy industry (e.g., fermented dairy products), leather industry, and tobacco industry. Thus, fermentation includes alcohol fermentation. Fermentation also includes anaerobic fermentations.

Fermenting can be accomplished by any organism suitable for use in a desired fermentation step. Suitable fermenting organisms are those that can convert DP1-3 sugars, especially glucose and maltose, directly or indirectly to the desired fermentation product (e.g., ethanol, propanol, butanol or organic acid). Fermenting can be effected by a microorganism, such as fungal organisms (e.g., yeast or filamentous fungi). The yeast can include strains from a Pichia or Saccharomyces species. The yeast can be Saccharomyces cerevisiae. Bacteria can also be used in a fermentation process. Bacteria include but are not limited to species from Acetobacter, engineered E. coli, Clostridium, Acidofilous or Lactobacter.

Fermenting can include contacting a mixture, including sugars from the reduced plant material, with yeast under conditions suitable for growth of the yeast and production of ethanol. In some embodiments, fermenting involves simultaneous saccharification and fermentation (SSF). The amount of yeast employed can be selected to effectively produce a desired amount of ethanol in a suitable time.

“Slurry tank” refers to any tank used to contain ground plant material combined with a liquid. A commercial slurry tank is a slurry tank used in a commercial production setting which may be a dry grind ethanol plant, a grain milling plant using a wet or dry milling process to mill corn grain or may be a food production plant that is combining ground plant flour with liquids in order to form a dough. A commercial slurry tank can be in an ethanol production facility that produces 10 million gallons of ethanol per year, 20 million gallons of ethanol per year, 30 million gallons of ethanol per year, 40 million gallons of ethanol per year, 50 million gallons of ethanol per year, 60 million gallons of ethanol per year, 70 million gallons of ethanol per year, 80 million gallons of ethanol per year, 90 million gallons of ethanol per year or 100 million gallons of ethanol per year, 150 million gallons of ethanol per year, 200 million gallons of ethanol per year, 250 million gallons of ethanol per year or more.

The term “hydrolysis” is defined as a chemical reaction or process in which a chemical compound is broken down by reaction with water. The starch digesting enzymes hydrolyze starch into smaller units as previously described.

Over the last few decades, several advances have been made to improve the ethanol yields and profitability of the dry grind process, including modifications in the production process [6], recovery of high-value co-products [5,7], use of advanced enzymes [8-9], and use of high-yield corn varieties [10]. In embodiments, the present invention provides new methods and compositions for improving ethanol yields by combined use of a new corn developed by transgenic technology, known as amylase corn, which transgenically produces an alpha-amylase in endosperm that is activated at high temperature and moisture [10-11] and a new engineered yeast (engineered to express glucoamylase (“superior yeast”).

In embodiments, due to high expression levels of alpha-amylase enzyme in the transgenic amylase corn, only a small amount of the transgenic amylase corn is mixed with conventional dent corn. Use of the transgenic amylase corn in a mix with conventional corn during the dry grind process can eliminate the need for external addition of exogenous alpha-amylase.

Similarly, in embodiments, a new engineered yeast, referred to as “superior yeast” herein, is an advanced strain of Saccharomyces cerevisiae that transgenically expresses glucoamylase and provides novel metabolic pathways for high ethanol yield by reducing glycerol production. Use of this transgenic yeast can eliminate or reduce the addition of exogenous glucoamylase enzymes during the SSF process, potentially improving the process efficiency, and reducing the overall ethanol production cost.

In embodiments, increasing the solid loadings during the dry grind process can be another approach to reduce the overall cost of ethanol production process. Using high solid slurries in a dry grind process can decrease the overall energy use and process cost by reducing load on downstream processing of ethanol and co-product recovery, and lowering the volumes handled by the processing equipment. However, the solid loadings during the ethanol process are generally restricted to 30 to 32% w/w due to high viscosities, and yeast stress by high glucose and ethanol concentrations [12-14]. High solid loadings can lead to higher final ethanol concentrations; however, low ethanol yields (Liters/metric ton or gallons/bushel) are observed because of strong ethanol inhibition [15]. In embodiments, simultaneous stripping off ethanol under vacuum during the SSF process is one of the potential approaches to reduce the ethanol inhibition and achieve high solid loadings [16]. In representative embodiments, with application of a vacuum, ethanol can be evaporated at the normal fermentation temperature without affecting the yeast activity. Some studies on ethanol and butanol production have concluded that fermentation efficiencies can be improved significantly by applying only few cycles of vacuum [12-13, 17].

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

In embodiments, the present invention provides strategies to reduce external exogenous enzyme requirements, e.g., during a dry grind process, and/or to improve ethanol yields at high-solid loadings. The fermentation characteristics of dent corn and “amylase mix corn” (transgenic corn expressing alpha-amylase mixed with conventional dent corn) were evaluated using a “superior yeast” (yeast expressing glucoamylase [GA]) at various loadings of glucoamylase enzyme (0, 25 and 50%), and the performance was compared with conventional yeast and exogenous glucoamylase used in the dry-grind process. The fermentation behavior of amylase mix corn using superior yeast was investigated using a vacuum flashing process to achieve high ethanol yields by reducing ethanol inhibition at high solid loadings.

In these examples, the fermentation of amylase corn (producing transgenically expressed alpha-amylase) using conventional yeast and no addition of exogenous alpha-amylase resulted in ethanol concentration of 4.1% higher as compared to control treatment (conventional corn using exogenous alpha-amylase). Conventional corn processed with exogenous alpha-amylase and superior yeast (producing glucoamylase) with no exogenous glucoamylase addition, resulted in an ethanol concentration similar to control treatment (conventional yeast with exogenous glucoamylase addition). The combination of amylase corn and superior yeast, required only 25% of the recommended glucoamylase dose to complete fermentation and achieve an ethanol concentration and yield similar to control treatment (conventional corn with exogenous alpha-amylase, conventional yeast and exogenous glucoamylase). Use of superior yeast with 50% GA addition resulted in similar increases in yield of approximately 7% for conventional or amylase corn compared with that of the control treatment. The combination of amylase corn, superior yeast and in situ ethanol removal resulted in a process that allowed complete fermentation of 40% slurry solids with only 50% of exogenous enzyme requirements and 64.6% higher ethanol yield compared with that of the conventional process.

### Example 1

### Materials and Methods

Amylase corn transgenically expressing alpha-amylase was obtained from a commercial seed company (Syngenta Biotechnology, Inc., Research Triangle Park, N.C.). Samples of conventional yellow dent corn and amylase corn were hand-cleaned and sieved using a 12/64″ (4.8 mm) sieve to remove broken corn and foreign materials. The cleaned corn was stored in a refrigerator at 4° C. until analysis. The moisture content in the corn was determined by drying samples in a hot air oven at 135° C. for 2 h (AACC International Approved Method 44-19.01) [18]. Starch content in the ground corn flour was determined using enzymatic assay (AACC International Approved Method 76-13.01) using the Total Starch Kit (Megazyme, Bray, Co. Wicklow, Ireland) [18].

The alpha-amylase and glucoamylase employed in this study were commonly-used commercial enzymes. The alpha-amylase enzyme has an activity of 6400 μmol maltose/min mL. The glucoamylase enzyme activity has been reported as 775 AGU/mL. Conventional active dry yeast (Ethanol Red) was obtained from the Fermentis-Lessaffre Yeast Corporation (Milwaukee, Wis.). The “superior yeast” (transgenically expressing glucoamylase) was provided by the Lallemand Biofuels & Distilled Spirits (Milwaukee, Wis.).

### Dry-Grind Process

The cleaned samples were ground in a laboratory scale hammer mill (model MHM4, Glen Mills, Clifton, N.J.) at 500 rpm and using a 0.5 mm screen. Conventional dent corn and amylase corn were ground separately and later mixed to form a 15% (by dry weight) amylase corn mixture, referred as “amylase mix corn.” All dry grind experiments were performed at 250 mL scale in 500 ml stainless steel reactors in triplicate. Ground corn was mixed with deionized (D.I.) water to make a slurry having 30% solids on dry basis. For the liquefaction of control samples (100% dent corn), the pH of the slurry was adjusted to 5.1 using 10 N sulfuric acid, and 25.7 μL of alpha-amylase was used per 100 g dry corn as per manufacturer's recommendations. The pH was not adjusted in the case of amylase corn mix, and no external alpha-amylase was added. The liquefaction was performed at 85° C. for 90 minutes using a Labomat incubator with continuous agitation (Labomat BFA-12, Werner Mathis AG, Switzerland). The heating and cooling time (heating and cooling rate of 3° C./min) were in addition to liquefaction time (90 min).

The pH of the liquefied slurry was adjusted to 4.8 using 10 N sulfuric acid for the SSF process. In control samples, yeast inoculum (2 mL), urea (0.4 mL of 50% w/v solution), and GA (56.3 μL/100 g dry corn) were added, and the slurry was fermented at 32° C. for 72 h in an automatic incubator (New Brunswick Innova 42R Inc/Ref Shaker, Eppendorf, Conn.) with continuous agitation at 150 rpm. Yeast inoculum was prepared by mixing 5 g of active dry yeast with 25 mL water and incubated at 32° C. for 20 minutes. SSF experiments using superior yeast were performed at three GA loadings (0, 25 and 50% of recommended dosage). The superior yeast was inoculated at the rate of 0.176 g per liter of slurry (˜50 μL for 250 mL slurry), as recommended by the manufacturer. Similar to the control experiments, urea solution was used as a nitrogen source, and the slurry was fermented at 32° C. for 72 h in an automatic incubator with continuous agitation at 150 rpm.

To monitor the fermentation, about 2 mL of sample was drawn at 0, 4, 8, 12, 24, 36, 48, and 72 h and centrifuged at 10,000 rpm (Eppendorf Centrifuge 5415 D, Eppendorf AG, Hamburg) for 10 minutes. The liquid was immediately filtered through 0.2 μm Acrodisc nylon syringe filters (Pall Life Sciences, Port Washington, N.Y.) into HPLC vials. The vials were frozen at −20° C. until further analyzed for sugar and ethanol content.

### Vacuum-Assisted Fermentation

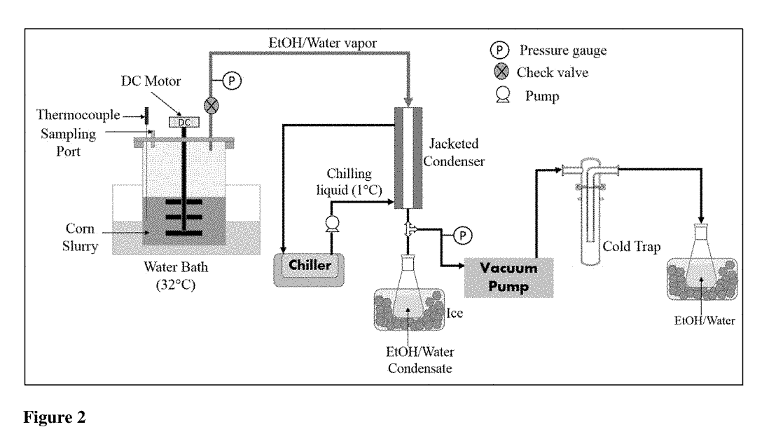

The vacuum-assisted fermentation experiments were performed using a lab scale modified vacuum-reactor system as shown in FIG. 2. It consists of a 3 L modified jacketed fermenter, modified to accommodate thermocouples, agitating motor with stirring blades, and a sampling port. A dry vacuum pump (DryFast model 2044, Welch, Niles, Ill.) was used to create the vacuum in the fermenter. The system has the ability to condense the evaporated ethanol and water vapors by passing those through a coiled condenser (5977-19, Ace Glass, Vineland, N.J.), with chilled liquid circulated at 1° C. The condensate was collected in a 250 mL conical flask kept under low temperature using ice. For other constructional and operational details of the system, please refer to Huang et al (2015) [17].

Slurry at 40% solids was prepared by mixing 500 g (dry basis) of 15% amylase mix corn with a calculated amount of D.I. water. The slurry was liquefied at 85° C. for 90 min. in multiple 500 mL stainless steel reactors using a Labomat incubator as described in the previous section. The liquefied slurry from multiple reactors was mixed in the 3 L fermenter, and pH was adjusted to 4.8 using 10 N sulfuric acid. The slurry was inoculated with 2 mL urea solution, 0.25 mL superior yeast and 140.8 μL of glucoamylase (50% of recommended dose for conventional yeast) and was incubated in a water bath set at 32° C. for 72 h. Vacuum pressure at 6.7 kPa (28 in Hg gauge) was applied for 1.5 h at 24, 36, 48, and 60 h of the fermentation. The vapors formed due to boiling of the slurry were condensed and collected in 250 mL conical flask. A sample was withdrawn from each condensate to determine the ethanol concentration using HPLC. For evaluation of the fermentation profile, about 2 mL of sample was withdrawn at 0, 4, 8, 12, 24, 36, 48, and 72 h of fermentation from the slurry and prepared for HPLC analysis as explained earlier. The samples were also withdrawn after the application of vacuum and analyzed for the sugar and alcohol concentrations.

### Sample Analysis (HPLC Analysis)

The fermentation samples were analyzed by high-performance liquid chromatography (HPLC; Waters Corporation, Milford, Mass.) using an ion-exclusion column (Aminex HPX-87H, Bio-Rad, Hercules, Calif.). The mobile phase was 0.005M sulfuric acid at 50° C. with a flow rate of 0.6 mL min⁻¹. For each sample, a 5 μL injection volume was used with a run time of 30 min. The amounts of sugars, alcohols and organic acids were quantified using a refractive index detector and using multiple standards.

### Ethanol Yields and Conversion Efficiency

Theoretical ethanol yields were estimated using equations 1 and 2, based on the starch content and free glucose of the corn, assuming complete starch conversion and 100% fermentation efficiency.

$$\begin{matrix}{V}_{ma x \_ EtOH} = \frac{{W}_{C} * ( 1 - {MC}_{C} ) * [ ( S * 1.11 * 0.511 ) + ( G * 0.511 ) ]}{{ρ}_{EtOH}} & ( 1 ) \\ {E}_{Th \_ EtOH} = \frac{{V}_{ma x \_ EtOH}}{{W}_{C} * ( 1 - {MC}_{C} )} & ( 2 )\end{matrix}$$

Where, V_{max EtOH} is the maximum possible volume of ethanol, mL; W_{C} is weight of the corn, g; MC_{C} is the moisture content in the corn; S is starch content; G is free glucose in corn; ρ_{EtOH} is density of ethanol, 0.789 g/mL; E_{Th}_{_}_{EtOH} is theoretical ethanol yield, L/kg dry corn; 1.11 is the gains during hydrolysis of starch; 0.511 is glucose to ethanol conversion ratio, kg/kg.

Actual ethanol yields were determined by calculating liquid volume in the final slurry after 72 h of fermentation. Weight of the final slurry was noted and a sample of the slurry was dried in a hot air oven at 105° C. until constant weight was achieved (˜24 h) to estimate the solid percent in the slurry. The actual ethanol yields were calculated using equations 3-5.

$$\begin{matrix}{W}_{L} = {W}_{slurry} * ( 1 - {Solids}_{Slurry} ) & ( 3 ) \\ {V}_{EtOH} = \frac{{W}_{L}}{{ρ}_{{H}_{2} O / EtOH}} * {C}_{EtOH} & ( 4 ) \\ {E}_{EtOH} = \frac{{V}_{EtOH}}{{W}_{C} * ( 1 - {MC}_{C} )} & ( 5 )\end{matrix}$$

Where, W_{L} is the weight of liquid in the fermented slurry, g; W_{slurry} is the weight of fermented slurry, g; Solids_{Slurry} is the solid fraction in the slurry; V_{EtOH} is the volume of ethanol produced, mL; ρ_{H}_{₂}_{O/EtOH} is the density of water-ethanol mixture (g/L) at final ethanol concentration; C_{EtOH} is the final ethanol concentration, mL/L; E_{EtOH} is the actual ethanol yield, L/kg.

Ethanol conversion efficiencies were calculated by dividing actual ethanol yields with the theoretical ethanol yield (Eqn. 6).

$${η}_{EtOH} = \frac{{E}_{EtOH}}{{E}_{Th \_ EtOH}} * 100 \qquad ( 6 )$$

### Statistical Analysis

The final ethanol concentrations, ethanol yields, starch to ethanol conversion efficiencies, and final glycerol concentrations during various treatments were statistically compared using analysis of variance and Fisher's least significant difference (SAS version 9.3). The level selected to show the statistical significance in all cases was 5% (P<0.05).

### Example 2

### Comparison of Yellow Dent Corn and Amylase Mix Corn

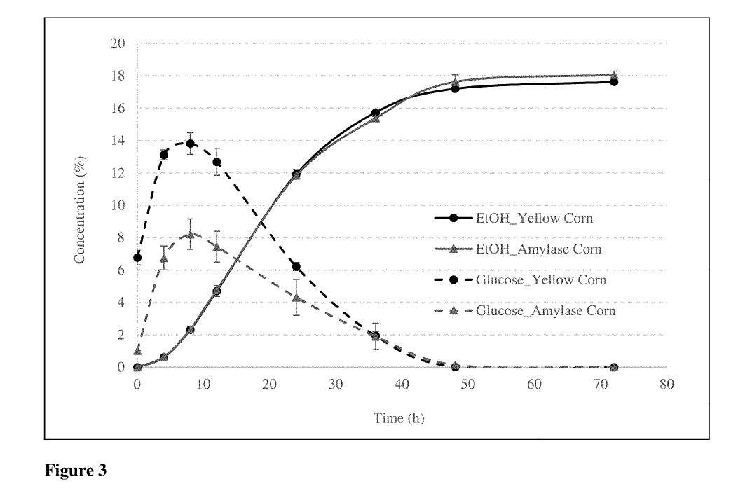

Ethanol and glucose concentration profiles during fermentation of yellow dent corn and amylase mix corn are illustrated in FIG. 3. After 72 h of fermentation, average final ethanol concentrations for dent corn and amylase mix corn were 17.62 and 18.05% (v/v) respectively. The small increase in final ethanol concentration for amylase corn could be due to relatively lower glucose inhibition. The peak glucose concentrations for yellow dent corn were much higher (13.8%) compared to that from using amylase corn mix (8.22%). The ethanol yield from amylase corn mix was calculated 0.444 L/kg dry corn (2.98 gal/bu), which was 4.1% higher than that of dent corn. Most of the fermentation was complete in 48 h for both cases, observed by the small (<0.25%) amounts of residual glucose, maltose, and maltotriose (Table 1). The results indicated that 15% addition of amylase corn mixed with conventional corn can eliminate the need for exogenous liquefaction enzyme that is currently used in the dry-grind process.

### Performance of Superior Yeast

SSF of Conventional Corn with Superior Yeast

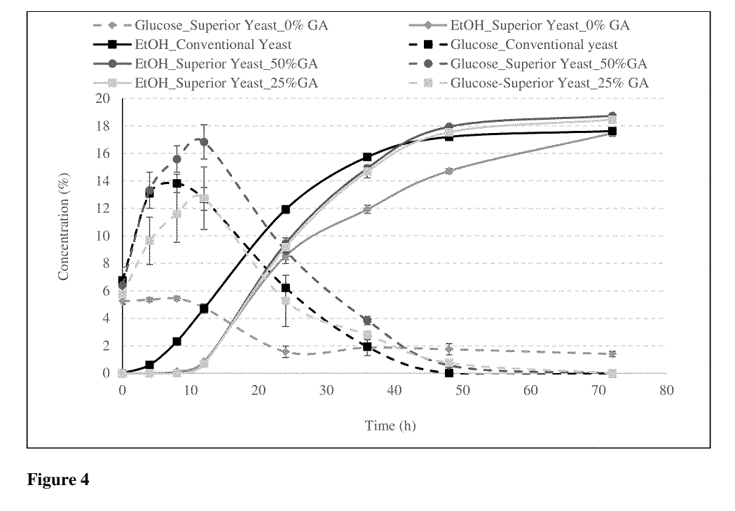

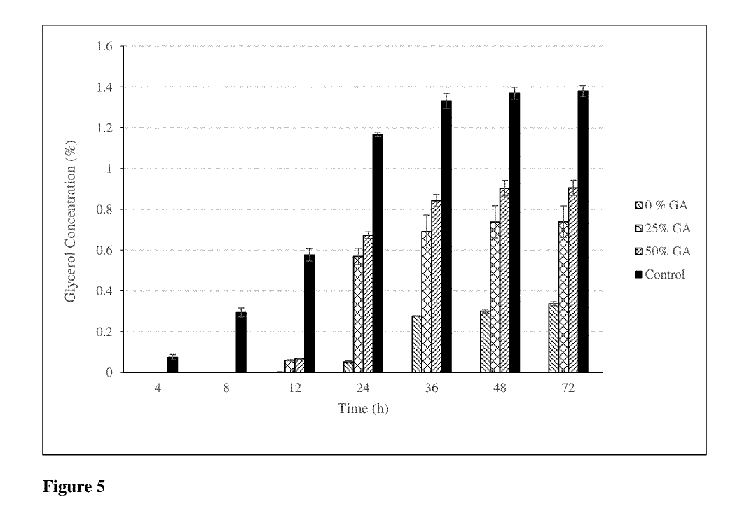

The ethanol and sugar production profiles during fermentation of conventional corn using conventional yeast at 100% GA loading and superior yeast with various glucoamylase loadings are illustrated in FIG. 4. Use of superior yeast even without any addition of glucoamylase (0%) resulted in a similar final ethanol yield as the control (p>0.05), indicating that superior yeast has sufficient GA expression to achieve similar ethanol profiles as with the control (Table 2). One important factor for these results could be lower substrate inhibition of the yeast. The glucose concentrations were relatively low throughout (1.41-5.24% w/v) the fermentation process for 0% GA loading, indicating relatively slow conversion of dextrins to glucose, which was simultaneously converted to ethanol by yeast. During the initial 12 h of SSF, fermentation rates were very low for superior yeast for all GA loadings. Ethanol concentrations were observed to be higher by addition of 25% and 50% GA along with the superior yeast (FIG. 4). Another major reason for high ethanol production using superior yeast was lower levels of glycerol production during the fermentation process. The glycerol production was lower in all cases of superior yeast compared to that for conventional yeast (FIG. 5). Glycerol production is considered an indicator of yeast stress, and typically about 1.2-1.5% glycerol concentrations are observed in dry-grind ethanol fermentations [19-20]. In this study, for the superior yeast, a maximum glycerol concentration of 0.91% was observed at 50% GA loading, which was still about 35% less than that of control. In the case of superior yeast used without any addition of GA, a final glycerol concentration of only 0.34% was observed, which was about 75% less than that of control. The ethanol yields of dent corn fermented using superior yeast were in the range of 0.423-0.461 L/kg of dry corn (2.84-3.1 gal/bu). Maximum starch to ethanol conversion efficiency of 88.5% was observed in the case of 50% GA addition (Table 2). Peak glucose concentration was maximum for superior yeast with 50% GA addition. In the case of superior yeast, it was observed that the peak glucose was observed at 12 h instead of at 8 h as in the control, indicating relatively slow initial saccharification during SSF.

SSF of Amylase Mix Corn with Superior Yeast

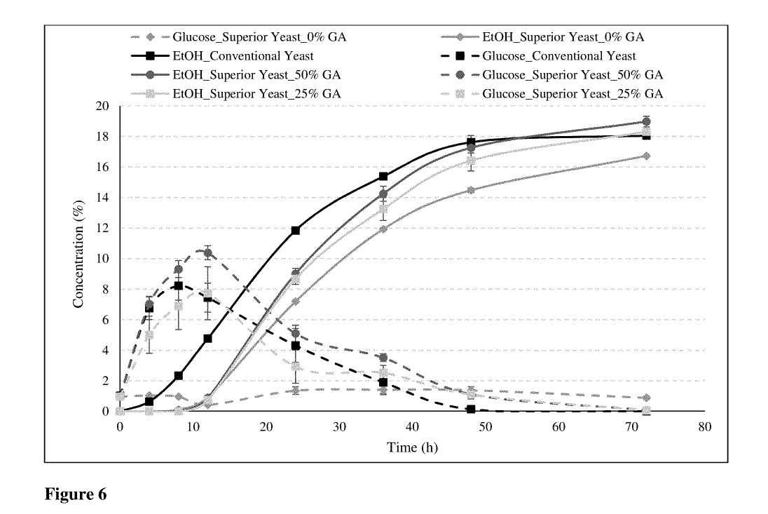

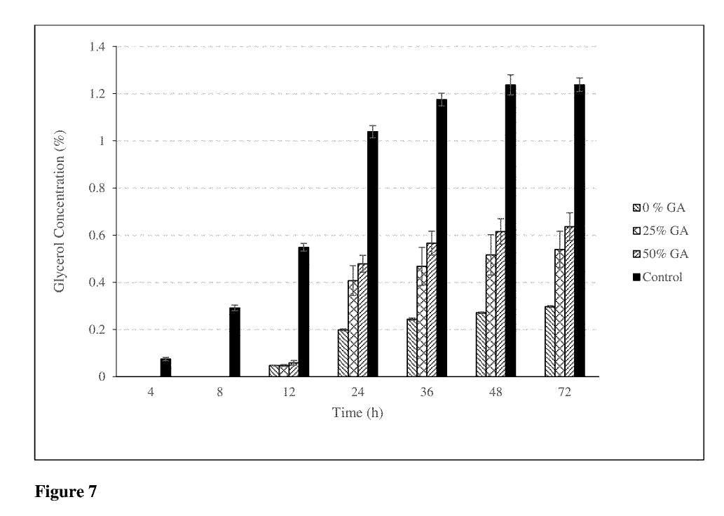

The performance of superior yeast with amylase corn mix was similar to that of conventional corn. The peak glucose during amylase corn mix fermentation using superior yeast was observed at 12 h instead of at 8 h as in case of control, indicating relatively slow conversion (FIG. 6). Compared to those for conventional corn, overall glucose concentrations were lower for all GA loadings with amylase corn mix, as observed with the conventional yeast also. Amylase corn mix fermented using superior yeast was considered as the control for these experiments. The final ethanol concentration using superior yeast without any addition of GA was about 7.3% lower than that of control. Addition of only 25% GA resulted in high ethanol concentration (18.31%), similar to that of control (18.05%, using conventional yeast). These results indicate that combined use of amylase corn and superior yeast in the dry grind process reduced the total external enzyme (alpha-amylase and glucoamylase) addition by more than 80%, which would significantly reduce the processing cost. An ethanol concentration as high as 18.7% was observed at 50% GA addition along with superior yeast use. At this GA loading, ethanol yield was estimated as 0.454 L/kg dry corn (3.05 gal/bu), about 2.35% higher than that of control. Ethanol conversion efficiencies for amylase mix corn using superior yeast ranged from about 77.57% to 87.01%. Similar to the case of dent corn, lower levels of glycerol production could have resulted in higher ethanol yields when using superior yeast (FIG. 7). In the case of 25% GA addition with use of superior yeast, final glycerol concentration (0.54%) was 56.4% lower than that for conventional yeast (1.24%). Maximum glycerol concentration of 0.64% was observed at 50% GA loading, and was about 49% less than that of control. The glycerol concentrations for amylase corn in all cases were lower than that of conventional corn.

### Effect of Solid Loadings

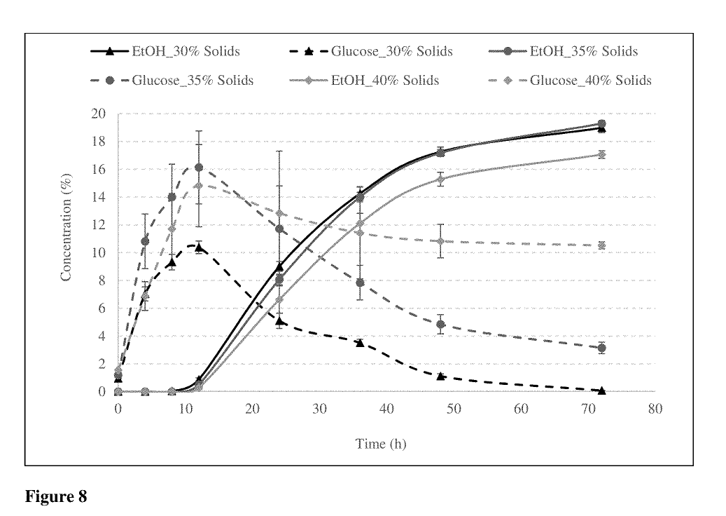

To examine the performance of superior yeast at high solid loadings, amylase mix corn was also liquefied at 35% and 40% solids, and the slurry was fermented using superior yeast with 50% GA addition. FIG. 8 illustrates the glucose and ethanol concentrations during fermentation at these solid loadings compared to those at 30% solids. Although the final ethanol concentration at 35% solids (19.28%) was higher than that at 30% solids (18.97%), about 3.14% glucose remained unconverted after 72 h of fermentation as compared with complete conversion at 30% solids. Final ethanol concentration at 40% solids was lower (17.1%) than both 30% and 35% solids, and 10.5% of glucose remained unconverted. The ethanol yields for 35% and 40% solids were 0.358 and 0.268 L/kg dry corn (2.40 and 1.76 gal/bu), respectively, which were 21.14% and 42.0% lower than that at 30% solids. High viscosities and yeast stress due to high glucose and ethanol concentration reduces the yeast productivity and results in lower ethanol yields. In addition, in this study the peak glucose concentrations for 35% and 40% solids were 1.55 and 1.42 times higher than that at 30% solids.

### In Situ Ethanol Removal During High Solid SSF

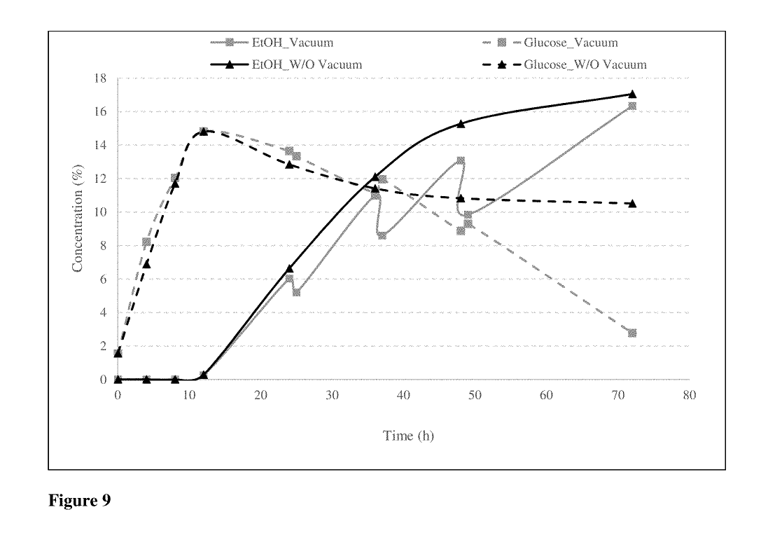

Simultaneous stripping of ethanol during the SSF process can reduce the ethanol inhibition and improve yeast activity. Experiments were performed to identify the suitable vacuum conditions (vacuum cycles and their frequency) for fermentation at 40% solids. Application of vacuum for 1 h at 24, 36, and 48 h during fermentation resulted in relatively high ethanol yields; however, there was still about 2.78% of the glucose left unconsumed at the end of fermentation (FIG. 9). Even after removal of significant amounts of ethanol during the fermentation process, the final ethanol concentrations were close to that of conventional fermentation (16.33 vs. 17.05% v/v). Ethanol yield at 40% solids was calculated as 0.38 L/kg (2.55 gal/bu), about 44% higher than that of conventional fermentation.

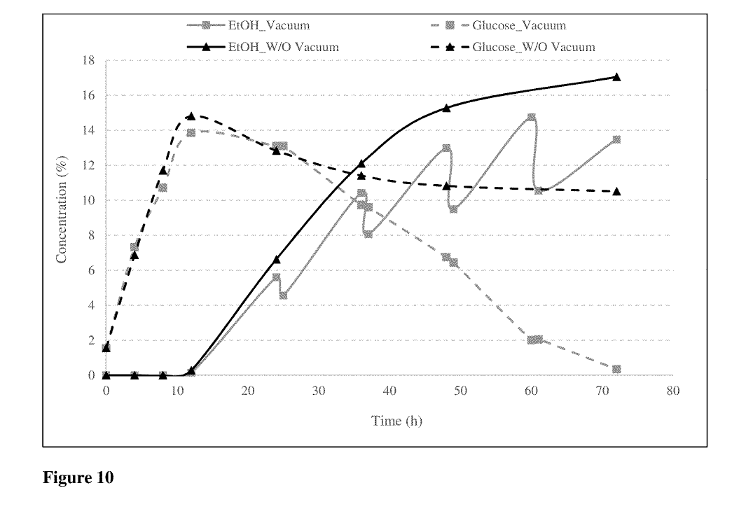

To further improve the fermentation efficiency, another vacuum cycle was added at 60 h and the vacuum time was increased to 90 minutes. Application of vacuum for 1.5 h at 24, 36, 48 and 60 h during SSF process resulted in complete fermentation compared to 10.5% residual sugars in case of conventional process (FIG. 10). After vacuum application for 90 minutes, the ethanol concentrations dropped in the range of 10.4-41.9 mL/L, depending upon the ethanol concentrations at the start of vacuum application. The ethanol drop was higher than those in previous experiments with 60 minute vacuum application (8.2-32.3 mL/L). The final ethanol yield with 82.89% ethanol conversion efficiency was estimated as 0.433 L per kg dry corn, which was about 1.65 times that for the conventional fermentation at 40% solids and only 4.6% lower than that at 30% solids. Similar results were observed by Shihadesh et al (2014) for dent corn ethanol production using granular starch hydrolyzing enzymes (GSHE) and conventional dry active yeast [13]. The ethanol yields at 40% solids fermentation with vacuum application produced similar ethanol yields as those of 30% solids during conventional fermentation.

The ethanol concentrations in the collected condensates ranged from 42.23-71.75% (v/v), with an average of 57.1% (v/v). This concentrated ethanol solution can potentially be directly guided to the rectification column during the distillation process for ethanol recovery, which can significantly reduce the energy load on the beer column (P^{t} stage of the ethanol recovery process) and overall cost of the dry grind process.

Conventional dent corn and amylase mix corn were processed in a dry-grind process using superior yeast that expresses glucoamylase with a reduction in the addition of external enzyme. Inclusion of amylase corn at a concentration of only 15% in the corn mix was sufficient to eliminate the need for exogenous alpha-amylase addition during liquefaction and achieved similar fermentation profiles to the conventional process. For yellow dent corn, no significant differences were observed in the ethanol yields between the control treatment and treatment with superior yeast in the absence of external glucoamylase addition. Use of superior yeast can significantly reduce the glucoamylase requirement, improve ethanol yields and reduce glycerol production. The vacuum flashing process successfully removed ethanol from the fermentation broth, and resulted in complete sugar consumption for a 40% solids slurry. The ethanol yield of 2.9 gal/bu of dry corn with more than 80% ethanol conversion efficiency, was about 65% higher than that at 40% solids for conventional fermentation. This study provided valuable insights about the use of amylase corn transgenically expressing alpha-amylase and superior yeast transgenically expressing glucoamylase in the dry grind processing industry, and the application of vacuum-assisted fermentation to improve fermentation at high solids.

The foregoing is illustrative of the present invention, and is not to be construed as limiting thereof. The invention is defined by the following claims, with equivalents of the claims to be included therein.

### Abbreviations

C_{EtOH}, final ethanol concentration; DDGS, distillers dried grains with soluble; D.I., deionized; E_{EtOH}, actual ethanol yield; E_{Th}_{_}_{EtOH}, theoretical ethanol yield; G, free glucose in corn; GA, glucoamylase; GSHE, granular starch hydrolyzing enzymes; MC_{C}, moisture content of the corn; S, starch content; SSF, simultaneous saccharification and fermentation; SY, superior yeast; Solids_{Slurry}, solid fraction in the slurry; Vₘₐₓ_{_}_{EtOH}, maximum possible volume of ethanol; V_{EtOH}, volume of ethanol produced; W_{C}, weight of the corn; W_{slurry}, weight of fermented slurry; ρ_{EtOH}, density of ethanol; ρ_{H}_{₂}_{O/EtOH}, density of water-ethanol mixture.

### Tables

TABLE 1
Comparison of sugar concentrations during SSF Process among yellow corn
and amylase mix corn (means ± standard deviation of triplicate runs)

| | Yellow Corn | | | | Amylase mix Corn | | | |
|---|---|---|---|---|---|---|---|---|
| Time | Glucose | Maltotriose | Maltose | Fructose | Glucose | Maltotriose | Maltose | Fructose |
| (h) | (% w/v) | (% w/v) | (% w/v) | (% w/v) | (% w/v) | (% w/v) | (% w/v) | (% w/v) |
| 0 | 6.76 ± 0.45 | 1.72 ± 0.23 | 2.48 ± 0.28 | 0.15 ± 0.01 | 1.02 ± 0.02 | 1.22 ± 0.11 | 4.73 ± 0.3 | 0.13 ± 0.01 |
| 4 | 13.1 ± 0.31 | 1.02 ± 0.25 | 6.35 ± 0.22 | 0.44 ± 0.03 | 6.75 ± 0.74 | 2.97 ± 0.08 | 7.23 ± 0.48 | 0.40 ± 0.02 |
| 8 | 13.8 ± 0.67 | 0.01 ± 0.01 | 5.28 ± 0.33 | 0.41 ± 0.05 | 8.22 ± 0.94 | 1.52 ± 0.63 | 9.63 ± 0.37 | 0.33 ± 0.02 |
| 12 | 12.69 ± 0.83 | 0 | 3.19 ± 0.13 | 0.35 ± 0.03 | 7.44 ± 0.95 | 1.36 ± 2.35 | 8.96 ± 0.74 | 0.25 ± 0.02 |
| 24 | 6.21 ± 0.25 | 0.16 ± 0.02 | 0.26 ± 0.01 | 0.2 ± 0.02 | 4.31 ± 1.1 | 1.38 ± 0.33 | 1.65 ± 0.83 | 0.10 ± 0.02 |
| 36 | 1.93 ± 0.27 | 0.08 ± 0.002 | 0.20 ± 0.01 | 0.14 ± 0.01 | 1.9 ± 0.81 | 1.47 ± 0.26 | 0.19 ± 0.01 | 0.38 ± 0.52 |
| 48 | 0.01 ± 0.01 | 0.03 ± 0.003 | 0.15 ± 0.02 | 0.08 ± 0.002 | 0.14 ± 0.03 | 0.22 ± 0.06 | 0.18 ± 0.02 | 0.07 ± 0.01 |
| 72 | 0 | 0 ± 0.006 | 0.10 ± 0.02 | 0.08 ± 0.004 | 0 | 0.04 ± 0.002 | 0.14 ± 0.004 | 0.08 ± 0.01 |

TABLE 2
Ethanol yields and conversion efficiencies (means ± standard deviation of triplicate runs)*

| | Final Ethanol | Final Glycerol | Ethanol Yield | Conversion |
|---|---|---|---|---|
| Conditions | Concentration (%) | Concentration (%) | (gal/bu, dry basis) | Efficiency (%) |
| Conventional Corn_Conventional yeast | 17.62 ± 0.19 e | 1.38 ± 0.03 a | 2.86 ± 0.06 d | 81.97 ± 1.26 c |
| Conventional Corn_SY_0% GA | 17.46 ± 0.22 e | 0.38 ± 0.01 g | 2.84 ± 0.03 d | 81.30 ± 0.85 c |
| Conventional Corn_SY_25% GA | 18.45 ± 0.22 b c | 0.74 ± 0.08 d | 3.04 ± 0.03 a b | 87.07 ± 0.91 a |
| Conventional Corn_SY_50% GA | 18.73 ± 0.15 a b | 0.91 ± 0.04 c | 3.09 ± 0.02 a | 88.50 ± 0.55 a |
| 15% Amylase Corn_Conventional yeast | 18.05 ± 0.23 d | 1.24 ± 0.03 b | 2.98 ± 0.06 b c | 85.07 ± 1.80 b |
| 15% Amylase Corn_SY_0% GA | 16.73 ± 0.06 f | 0.30 ± 0.01 g | 2.72 ± 0.01 e | 77.57 ± 0.33 d |
| 15% Amylase Corn_SY_25% GA | 18.31 ± 0.18 c d | 0.54 ± 0.08 f | 2.96 ± 0.01 c | 84.50 ± 0.33 b |
| 15% Amylase Corn_SY_50% GA | 18.97 ± 0.35 a | 0.64 ± 0.06 e | 3.05 ± 0.04 a | 87.01 ± 1.26 a |

SY—Superior yeast
*Means followed by the same letter in one column are statistically not different (at P < 0.05

### REFERENCES

1. Prasad, A, Sotenko, M, Blenkinsopp, T, Coles, S R. Life cycle assessment of lignocellulosic biomass pretreatment methods in biofuel production. The International Journal of Life Cycle Assessment. 2016, 21(1):44-50.
2. da Costa Sousa, L, Chundawat, S P, Balan, V, Dale, B E. ‘Cradle-to-grave’ assessment of existing lignocellulose pretreatment technologies. Current Opinion in Biotechnology. 2009, 20(3):339-347.
3. RFA. 2015. Going Global—2015 Ethanol Industry Outlook Renewable Fuels Association. www.ethanolrfa.org/resources/publications/outlook. Renewable Fuels Association: Washington, D.C. Accessed on Jun. 15, 2016.
4. Bothast, R, Schlicher, M. Biotechnological processes for conversion of corn into ethanol. Applied Microbiology and Biotechnology. 2005, 67(1):19-25.
5. Somavat, P, Li, Q, de Mejia, E G, Liu, W, Singh, V. Coproduct yield comparisons of purple, blue and yellow dent corn for various milling processes. Industrial Crops and Products. 2016, 87:266-272.
6. Singh, V, Johnston, D B, Naidu, K, Rausch, K D, Belyea, R L, Tumbleson, M. Comparison of modified dry-grind corn processes for fermentation characteristics and DDGS composition. Cereal Chemistry. 2005, 82(2):187-190.
7. Rausch, K D, Belyea, R L. The future of coproducts from corn processing. Applied Biochemistry and Biotechnology. 2006, 128(1): 47-86.
8. Uthumporn, U, Zaidul, I S, Karim, A. Hydrolysis of granular starch at sub-gelatinization temperature using a mixture of amylolytic enzymes. Food and Bioproducts Processing. 2010, 88(1):47-54.
9. Wang, P, Singh, V, Xu, L, Johnston, D B, Rausch, K D, Tumbleson, M. Comparison of enzymatic (E-Mill) and conventional dry-grind corn processes using a granular starch hydrolyzing enzyme. Cereal Chemistry. 2005, 82(6):734-738.
10. Singh, V, Batie, C J, Aux, G W, Rausch, K D, Miller, C. Dry-grind processing of corn with endogenous liquefaction enzymes. Cereal Chemistry. 2006, 83(4):317-320.
11. Urbanchuk, J M, Kowalski, D J, Dale, B, Kim, S. Corn amylase: improving the efficiency and environmental footprint of corn to ethanol through plant biotechnology. AgBioForum. 2009, 12:149-54.
12. Shihadeh, J, Huang, H, Rausch, K, Tumbleson, M, Singh, V. Design of a vacuum flashing system for high-solids fermentation of corn. Transactions of the ASABE. 2013, 56(6): 1441-1447.
13. Shihadeh, J K, Huang, H, Rausch, K D, Tumbleson, M E, Singh, V. Vacuum Stripping of ethanol during high solids fermentation of corn. Applied Biochemistry and Biotechnology. 2014, 173(2):486-500.
14. Taylor, F, Kurantz, M J, Goldberg, N, McAloon, A J, Craig, J C. Dry-Grind Process for Fuel Ethanol by Continuous Fermentation and Stripping. Biotechnology Progress, 2010, 16(4), 541-547.
15. Wang, S, Ingledew, W, Thomas, K, Sosulski, K, Sosulski, F. Optimization of fermentation temperature and mash specific gravity for fuel alcohol production. Cereal Chemistry. 1999, 76(1):82-86.
16. Ramalingham, A, Finn, R. The vacuferm process: a new approach to fermentation alcohol. Biotechnology and Bioengineering. 1977, 19(4):583-589.
17. Huang, H, Qureshi, N, Chen, M H, Liu, W, Singh, V. Ethanol production from food waste at high solids content with vacuum recovery technology. Journal of Agricultural and Food Chemistry. 2015, 63(10): 2760-2766.
18. American Association of Cereal Chemists International. Approved Methods of Analysis, 11th ed. AACC International, St. Paul, 2010.
19. Murthy, G S, Townsend, D E, Meerdink, G L, Bargren, G L, Tumbleson, M E, and Singh, V. Effect of aflatoxin B1 on the dry-grind ethanol process. Cereal Chemistry. 2005, 82:302-304.
20. Russel, I. Understanding yeast fundamentals. in: The Alcohol Textbook: A Reference for the Beverage, Fuel and Industrial Alcohol industries, 4th Ed. K. A. Jacques, T. P. Lyons, and D. R. Kelsall, eds. 2003. Nottingham University Press: Nottingham, UK

We claim:

1. A method of making ethanol comprising the steps of:
a) combining milled plant material comprising at least about 0.1% of plant material from a transgenic plant expressing alpha-amylase with a liquid to form a mash;
b) hydrolyzing starch in the mash to form a liquefact;
c) fermenting the liquefact in the presence of a yeast expressing glucoamylase to form a beer; and
d) distilling the beer to produce ethanol.

2. The method of claim 1, wherein the transgenic plant expressing alpha-amylase is a transgenic corn plant comprising the genotype of corn event 3272.

3. The method of claim 2, wherein the plant material is corn grain.

4. The method of claim 2, wherein the milled plant material comprises an admix of about 0.1% to about 100% of the transgenic corn plant comprising the genotype of corn event 3272 and about 99.1% to 0% of plant material not comprising the genotype of corn event 3272.

5. The method of claim 2, wherein the milled plant material comprises at least about 5% to about 50%, optionally about 5% to about 25%, of the transgenic corn plant comprising the genotype of corn event 3272.

6. The method of claim 1, further comprising adding one or more starch-degrading enzymes selected from the group of alpha-amylase, pullulanase, alpha-glucosidase, glucoamylase, amylopullulanase, glucose isomerase, and combinations thereof.

7. The method of claim 6, wherein the amount of exogenous glucoamylase added is about 10% to about 60% of that added in a control method of making ethanol that does not comprise yeast expressing glucoamylase as in step (c).

8. The method of claim 2, wherein no exogenous alpha-amylase is added in addition to that present in the plant material from the transgenic corn plant comprising the genotype of corn event 3272.

9. The method of claim 1, wherein no exogenous glucoamylase is added in addition to that from the yeast expressing glucoamylase.

10. The method of claim 2, wherein the genotype of corn event 3272 comprises the nucleotide sequence set forth in SEQ ID NO: 1 (ctgacgcggc caaacactga) and/or the nucleotide sequence set forth in SEQ ID NO: 2 (cacaatatat tcaagtcatc).

11. The method of claim 1, wherein the yeast expressing glucoamylase is a Saccharomyces spp, Candida spp., Schizosaccharomyces spp., Endomycopsis spp., Kluyveromyces spp., Pichia spp., Hanseniaspora spp., Trichoderma spp., Thermomyces spp., or any combination thereof.

12. The method of claim 1, wherein the yeast expressing glucoamylase is Saccharomyces cerevisiae, Candida arabinofermentans, Candida shehatae, Candida utilis, Candida valida, Candida lyxosophila, Candida mogii, Candida santjackobensis, Candida succiphila, Candida sorboxylosa, Candida stellate, Candida tenuis, Candida veronae, Schizosaccharomyces pombe, Endomycopsis fibuligera, Kluyveromyces marxianu, Pichia mexicana, Pichia anomala, Pichia guilliermondii, Pichia stipitis, Hanseniaspora valbyensis Trichoderma reesei., Thermomyces lanuginosus, or any combination thereof.

13. The method of claim 1, wherein the yeast expressing glucoamylase is Saccharomyces cerevisiae.

14. The method of claim 1, wherein the liquid comprises water.

15. The method of claim 1, wherein hydrolyzing starch in the mash is performed at a pH of about pH 3.8 to about pH 5.0.

16. The method of claim 1, wherein the mash has a percentage of solids of about 35% to about 60%.

17. The method of claim 1, wherein the mash has a percentage of solids of at least about 37%.

18. The method of claim 1, wherein the mash has a temperature which does not exceed about 90° C.

19. The method of claim 1, wherein after step (a) and prior to hydrolyzing the starch in the mash to form a liquefact (step (b)), the method comprises allowing the mash to flow from initial combining to a heat exchanger at a rate which is at least 5% to at least 70% less than the rate of flow of beer from a beer well to a distiller.

20. The method of claim 19, wherein the method comprises allowing the mash to flow from initial combining to a heat exchanger at a rate which is at least 20% to at least 55% less than the rate of flow of beer from a beer well to a distiller.

21. The method of claim 1, wherein after step (a) and prior to hydrolyzing the starch in the mash to form a liquefact (step (b)), the method comprises allowing the mash to flow from initial combining to a fermenter at a rate which is at least 5% to at least 70% less than the rate of flow of beer from a beer well to a distiller.

22. The method of claim 21, wherein the method comprises allowing the mash to flow from initial combining to a fermenter at a rate which is at least 20% to at least 55% less than the rate of flow of beer from a beer well to a distiller.

23. The method of claim 21, further comprising the step of allowing the mash to flow out of a fermenter at a rate that is approximately the nameplate flow rate.

24. The method of claim 21, comprising allowing the mash to flow from initial combining to a fermenter at a rate which is at least 20% to at least 55% less than the nameplate rate of flow.


SEQUENCE LISTING (2 sequences)

SEQ ID NO: 1 - DNA, 20 bp, Artificial Sequence
  5′ junction sequence
ctgacgcggccaaacactga

SEQ ID NO: 2 - DNA, 20 bp, Artificial Sequence
  3′ junction sequence
cacaatatattcaagtcatc